(19) United States
(12) Patent Application Publication
(10) Pub. No.: US 2026/0290582 A1
(43) Pub. Date: Sep. 24, 2026

(54) SYSTEM AND METHOD FOR DETECTION OF PREMATURE VENTRICULAR CONTRACTIONS ARRHYTHMIAS BASED ON INTERNET-OF-THINGS

(71) Applicant: NAJRAN UNIVERSITY, Najran (SA)

(72) Inventors: Mana Saleh AL RESHAN, Najran (SA); Qurat-ul-Ain MASTOI, Bristol (GB); Mohammed HAMDI, Najran (SA); Samar M. ALQHTANI, Najran (SA); Adel SULAIMAN, Najran (SA); Hani ALSHAHRANI, Najran (SA); Asadullah SHAIKH, Najran (SA); Darakhshan SYED, Karachi (PK)

(73) Assignee: NAJRAN UNIVERSITY, Najran (SA)

(21) Appl. No.: 19/082,988

(22) Filed: Mar. 18, 2025

(51) Int. Cl. G16H 50/20; A61B 5/00; G16H 40/67

(52) U.S. Cl. CPC G16H 50/20; A61B 5/0006; G16H 40/67

(57) ABSTRACT

A system and method for performing Internet-of-Things (IoT)-based detection of Premature Ventricular Contractions (PVC) arrhythmias includes electrocardiography (ECG) signal acquisition circuitry attached to a patient's body to acquire an ECG signal from the patient. The system includes IoT-based signal transmission circuitry using IoT communication to transmit the ECG signal to a Fog-network-based signal processing circuitry. The Fog-network-based signal processing circuitry uses a Fog network to apply a modified Pan and Tompkins algorithm to extract ECG features in frequency domain and time domain from the ECG signal; determine whether the patient has PVC arrhythmias, based on the extracted ECG features, provide an alert to the patient and a healthcare team upon determining that the patient has the PVC arrhythmias. The extracted ECG features include QRS complex durations, R-peaks, R-R intervals, an average of the R-R intervals, and an average deviation of the R-R intervals.

### BACKGROUND

### Technical Field

The present disclosure is directed to biomedical engineering and health informatics, and more particularly, to systems and methods for detection and classification of cardiac arrhythmias, such as premature ventricular contractions (PVC), using Machine Learning (ML) algorithms, Internet of Medical Things (IoMT), and advancements in signal processing.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Increasing global population and escalating healthcare costs have become significant challenges for citizens and healthcare authorities. A viable solution to alleviate burden on hospitals is to provide compassionate care to patients at home, thus reducing pressure on the hospitals caused by a rising number of patients. Additionally, the healthcare sector is facing challenges with a low survival rate associated with sudden cardiac events. While technology offers potential solutions for real-time detection, not all medical institutions are equipped to handle such emergency situations. Specialized treatments often require hospital stays of one or two days, with continuous monitoring by an expert. However, a major challenge in healthcare today is the lack of comprehensive health data and patient history provided by users.

The Internet of Things (IoT) technique can significantly enhance various services within smart healthcare, including telemedicine, elderly care, behavioral health monitoring, remote surveillance, treatment adherence monitoring, and resource management in healthcare facilities. These applications are expected to play a critical role in the future of healthcare. The integration of web services, electronic (e)-health, and smartphone communication presents numerous opportunities. Recent technological advancements have enabled wireless technologies to collaborate with healthcare systems, offering benefits that extend beyond the limitations of traditional healthcare infrastructures. These benefits include improved service quality and faster medical responses. In particular, a process of capturing real-time metrics and variables has been simplified by the Internet of Medical Things (IoMT). One such component is wearable health devices that allow remote monitoring of the patients during specific clinical events. The IoMT enhances healthcare efficiency by allowing the patients to access their data through online platforms or mobile phones.

The implementation of IoMT in the healthcare field can significantly lower mortality rates by improving an early detection and intervention of life-threatening conditions such as heart disease. Telemedicine has already proven effective in reducing mortality by decreasing a need for hospital-based admissions for the patients with pulmonary, heart, and neurological conditions. Cardiovascular disease remains a leading cause of death worldwide. An exciting approach to studying heart conditions involves artificial intelligence algorithms that classify heartbeats, identify arrhythmias, and predict cardiac diseases. Many studies have applied feature extraction and classification techniques to improve Electrocardiogram (ECG) monitoring for heart disease detection, particularly in arrhythmia diagnosis. Additionally, Bayesian classification methods for detecting Premature Ventricular Contraction (PVC) arrhythmias have been proposed that offer a robust and simple solution through probability distribution. However, these methods often lack accuracy in PVC heartbeat classification.

Further, a Pan and Tompkins algorithm is used as a benchmark, and QRS complex and R-peak durations are identified, even though the complexity of the algorithm is high. However, to accurately classify the PVC arrhythmias, additional relevant features are necessary. Many approaches have failed to extract critical features for PVC detection, particularly when examining inter-patient variability in ECG rhythms across different patterns. Additionally, inaccurate thresholds caused by interference often arise with a conventional adaptive threshold method employed by the Pan and Tompkins algorithm. Accurate prediction of the PVC arrhythmias requires considering multiple historical data points, yet many solutions have failed to focus on R-peak-related features. Misclassifications in real-world applications can be dangerous, as they fail to capture critical aspects of ECG data.

U.S. Patent Publication US20240306911A1 discloses a cardiac event monitoring system that applies ECG morphological analysis to detect abnormal cardiac events. A wearable cardiac sensing device acquires data, which is transmitted to a remote server using IoT protocols. The system filters out baseline drifting, high-frequency noise, and 50/60 Hz interferences, and identifies ECG features like P wave, the QRS complex, and T wave using a rhythm classifier based on the Pan-Tompkins algorithm. However, the reliance on the Pan-Tompkins algorithm for QRS detection can result in inaccuracies due to interference or noise, and the system may not effectively address variations in the ECG data across different patients, leading to potential misclassification of the cardiac events.

Indian Patent Publication IN202131020960A describes a system and method for detecting PVC using a machine learning algorithm. U.S. Patent Publication US20240138743A1 describes a system for detecting PVC using an artificial intelligence model. Chinese Patent Publication CN112686091B describes a two-step arrhythmia classification method based on deep neural network. However, each of the aforementioned references has limitations that restrict their broader applicability in the context of real-time PVC detection. These references fail to address the complexities of patient-specific variations in the ECG data, leading to potential inaccuracies in the PVC detection. Furthermore, they rely on traditional algorithms and pre-defined noise removal techniques that may not be adaptable to varying types of interference or individual patient conditions. Their centralized architecture and dependency on static feature extraction methods limit the flexibility and scalability needed for real-time, dynamic healthcare monitoring. Therefore, these existing solutions lack the precision and adaptability to provide reliable, real-time PVC detection in diverse healthcare environments.

Accordingly, one object of the present disclosure is to provide a system and method for detecting PVC arrhythmias and increasing the precision, effectiveness, and affordability of cardiac arrhythmia diagnostics using wearable health monitoring gadgets, remote patient monitoring mechanisms, and real-time data processing for medical applications.

### SUMMARY

In an exemplary embodiment, a system for performing Internet-of-Things (IoT)-based detection of Premature Ventricular Contractions (PVC) arrhythmias is disclosed. The system includes electrocardiography (ECG) signal acquisition circuitry. The system further includes IoT-based signal transmission circuitry. The system further includes IoT-based signal transmission circuitry. The ECG signal acquisition circuitry is configured to be attached to a patient's body to acquire an ECG signal from the patient. The IoT-based signal transmission circuitry is configured to use IoT communication to transmit the ECG signal to Fog-network-based signal processing circuitry. The Fog-network-based signal processing circuitry is configured to use a Fog network to apply a modified Pan and Tompkins algorithm to extract, from the ECG signal, a plurality of ECG features in frequency domain and time domain. The Fog-network-based signal processing circuitry is further configured to use the Fog network to determine whether the patient has PVC arrhythmias, based on the extracted plurality of ECG features. The Fog-network-based signal processing circuitry is further configured to use the Fog network to provide an alert to the patient and a healthcare team upon determining that the patient has the PVC arrhythmias. The extracted plurality of ECG features includes QRS complex durations, R-peaks, R-R intervals, an average of the R-R intervals, and an average deviation of the R-R intervals.

In another exemplary embodiment, a method for performing Internet-of-Things (IoT)-based detection of Premature Ventricular Contractions (PVC) arrhythmias is disclosed. The method includes via electrocardiography (ECG) signal acquisition circuitry attached to a patient's body, acquiring an ECG signal from the patient. The method further includes via IoT-based signal transmission circuitry, using IoT communication to transmit the ECG signal to Fog-network-based signal processing circuitry. The method further includes, via the Fog-network-based signal processing circuitry, applying a modified Pan and Tompkins algorithm to extract, from the ECG signal, a plurality of ECG features in frequency domain and time domain. The method includes, via the Fog-network-based signal processing circuitry, determining whether the patient has PVC arrhythmias, based on the extracted plurality of ECG features. The method includes, via the Fog-network-based signal processing circuitry, provide an alert to the patient and a healthcare team upon determining that the patient has the PVC arrhythmias. The extracted plurality of ECG features includes QRS complex durations, R-peaks, R-R intervals, an average of R-R intervals, and an average deviation of R-R intervals.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A illustrates a block diagram of a system for performing Internet-of-Things (IoT)-based detection of Premature Ventricular Contractions (PVC) arrhythmias, according to certain embodiments.

FIG. 1B illustrates an exemplary system workflow architecture, according to certain embodiments.

FIG. 1C illustrates a block diagram showing stages of processing an ECG signal performed by a Fog network using a modified Pan and Tompkins algorithm, according to certain embodiments.

FIG. 1D illustrates an architecture of Cyclic Echo State Network (CESN), according to certain embodiments.

FIG. 2 illustrates a flowchart of a method for performing the IoT-based detection of the PVC arrhythmias, according to certain embodiments.

FIG. 3 is an illustration of a non-limiting example of details of computing hardware used in a computing system, according to certain embodiments.

FIG. 4 is an exemplary schematic diagram of a data processing system used within the computing system, according to certain embodiments.

FIG. 5 is an exemplary schematic diagram of a processor used with the computing system, according to certain embodiments.

FIG. 6 is an illustration of a non-limiting example of distributed components which may share processing with a controller, according to certain embodiments.

### DETAILED DESCRIPTION

In the drawings, like reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a system and method for real-time Premature Ventricular Contractions (PVC) detection and classification using Electrocardiography (ECG) data and improving early detection and intervention of life-threatening cardiac events. Conventional methods for detecting the PVCs often rely on basic algorithms or standard thresholds, which may not account for variability in patient-specific ECG data and are prone to inaccuracies.

The present disclosure employs a machine learning-based approach for feature extraction and classification, offering a more accurate and adaptive solution for the PVC detection. The system processes the ECG data using advanced techniques such as noise removal, baseline drifting, and discrete wavelet transform, followed by the feature extraction from QRS complex to improve classification precision. By integrating Internet of Things (IoT) protocols and enabling real-time monitoring, the system provides actionable insights that can be accessed remotely, enhancing patient care and minimizing risks associated with undetected PVCs. Furthermore, scalability and adaptability of the system make it suitable for integration with existing healthcare infrastructure, ensuring effective real-time decision-making for healthcare professionals.

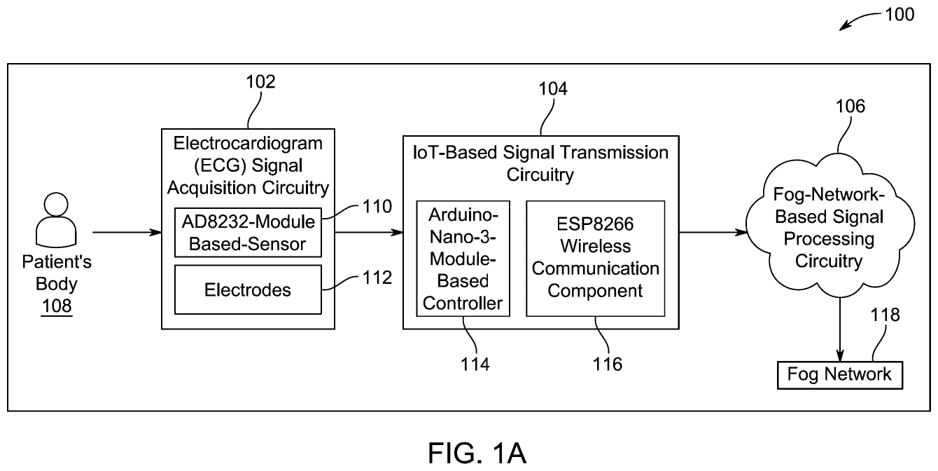

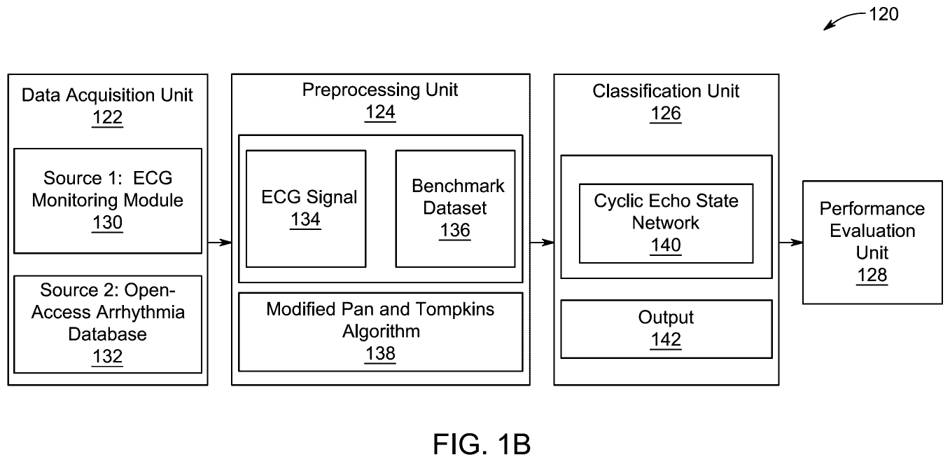

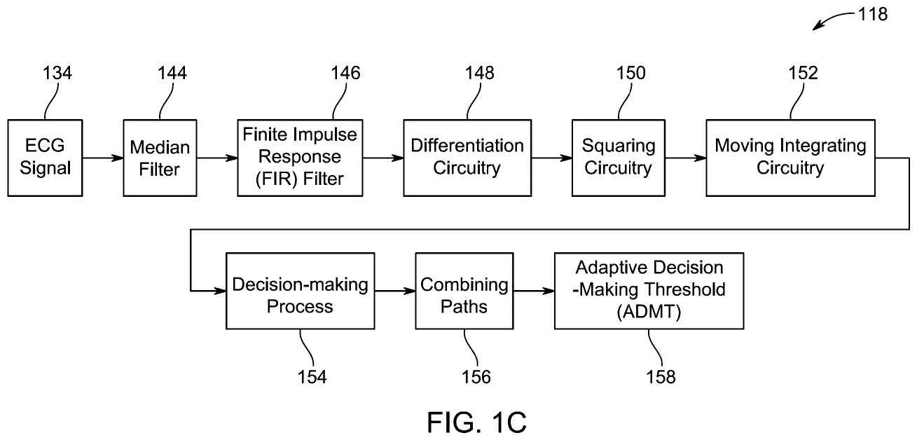

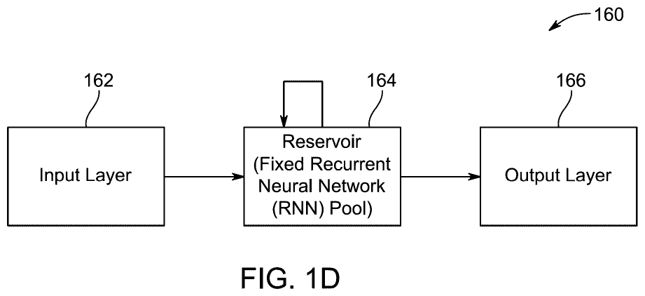

FIG. 1A illustrates a block diagram of a system 100 for performing Internet-of-Things (IoT)-based detection of PVC, according to certain embodiments. As used herein, the term “PVC” refers to a type of irregular heartbeat that originates in ventricles (main pumping chambers) of a heart. The PVC may cause a sensation of a skipped heartbeat or an extra heartbeat and is often detected on an ECG as an irregular and wide QRS complex. As used herein, the term “QRS complex” refers to a component of the ECG that represents electrical depolarization of the ventricles (i.e., a process by which ventricular muscle cells of the heart undergo a change in an electrical state, triggering a heart muscle to contract). The QRS complex is divided into three distinct parts such as Q wave (i.e., a small downward deflection that indicates initial depolarization of interventricular septum), R wave (i.e., a sharp upward deflection representing a main depolarization wave as it spreads through the ventricles) and S wave (i.e., a downward deflection following the R wave, indicating a final phase of ventricular depolarization at a base of the heart). In an embodiment, the system 100 may be implemented as a Fully Automated system for PVC Arrhythmia Classification (FAPAC) that provides a robust and machine learning-enabled approach for detecting the PVC with improved accuracy. In an embodiment, the machine learning-enabled approach may be used in combination with an Internet of Medical Things (IoMT) based framework to improve processing of an ECG signal 134 (as shown in FIG. 1B). In an embodiment, the ECG signal 134 may be considered as a preprocessed, normalized. and processed ECG signal. As used herein, the “Internet of Medical Things (IoMT) based framework” refers to a network of connected medical devices such as wearable ECG monitors, that communicate the ECG signal 134 in real-time to healthcare systems.

The system 100 includes ECG signal acquisition circuitry 102, IoT-based signal transmission circuitry 104, and Fog-network-based signal processing circuitry 106.

The ECG signal acquisition circuitry 102 is configured to be attached to a patient's body 108 to acquire the ECG signal 134 from a patient. As used herein, the “ECG signal 134” refers to an electrical representation of the activity of the heart over time, recorded from a surface of the patient's body 108. In an exemplary embodiment, the ECG signal acquisition circuitry 102 is designed to capture and process weak electrical signals produced by the heart. As used herein, the term “weak electrical signals produced by the heart” refers to low-amplitude electrical signals generated by the heart's electrical activity during each cardiac cycle. The ECG signal acquisition circuitry 102 is configured to amplify the captured weak electrical signals and filter out noise (e.g., baseline drifting, high-frequency noise, powerline interference) to provide a clean representation of the ECG of the patient.

The ECG signal acquisition circuitry 102 includes, for example, an AD8232-module-based sensor 110 (manufactured by Analog Devices, Inc., One Analog Way Wilmington, MA 01887, United States of America) and electrodes 112. Although the AD8232 module is shown to be used in the specification, one can appreciate ECG module heart rate monitor of any other make or manufactured by any other company can be used. The AD8232-module-based sensor 110 may be a compact and efficient ECG signal acquisition unit configured to monitor the electrical activity of the heart. In an embodiment, the AD8232-module-based sensor 110 may work in conjunction with the electrodes 112 to capture and process the ECG signal 134.

The electrodes 112 may be attached to limbs of the patient to detect the weak electrical signals of the heart. In an exemplary embodiment, the AD8232-module-based sensor 110 includes connectors for attaching three electrodes 112 such as, a Right Arm (RA) electrode, a Left Arm (LA) electrode, and a Right Leg (RL) electrode. The RA electrode may be positioned on the right arm of the patient and serve as a positive input electrode. The LA electrode may be positioned on the left arm of the patient and serve as a negative input electrode. The RL electrode may be positioned on the right leg of the patient and serves as a reference (ground).

In an exemplary embodiment, the electrodes 112 may create a conductive interface with the skin of the patient by using an attaching means (e.g., gel or adhesive). The electrodes 112 attached to the limbs of the patient may detect the electrical activity of the heart by picking up electrical impulses (i.e., the weak electrical signals) generated during depolarization and repolarization of the heart muscles. The electrical impulses may travel from the heart to the electrodes 112 through the skin, where the electrical impulses are converted into voltage signals (i.e., the electrical impulses are captured as small voltage differences by the electrodes 112).

In an embodiment, the AD8232-module-based sensor 110 and the electrodes 112 may be connected through wires. In such embodiment, the wires may act as conduits, ensuring minimal signal loss and interference during transmission of the ECG signal 134. In an embodiment, the wires may serve as a medium to transmit the weak electrical signals from the electrodes 112 on the skin to input pins of the AD8232-module-based sensor 110, where the weak electrical signals are processed and displayed as the ECG signal 134. In an embodiment, the AD8232-module-based sensor 110 may have input ports for receiving a positive signal (i.e., LA) of the weak electrical signals detected by the electrodes 112, a negative signal (i.e., RA) of the weak electrical signals detected by the electrodes 112, and a reference baseline (i.e., RL) for the ECG signal 134.

The AD8232-module-based sensor 110 may be configured to receive, amplify, and filter the weak electrical signals. In an exemplary embodiment, the AD8232-module-based sensor 110 may be integrated with an instrumentation amplifier that amplifies the weak electrical signals of the heart received from the electrodes 112. The instrumentation amplifier may ensure high gain (i.e., increases a signal strength), high Common Mode Rejection Ratio (CMRR) (i.e., filter out noise and interference, coming from environment or patient movements), minimal distortion (i.e., retains integrity of the ECG signal 134), and so forth.

Further, in an embodiment, the AD8232-module-based sensor 110 may also have built-in analog filters that prepare the ECG signal 134 for further processing. The analog filters may be high-pass filters (that remove baseline drifting caused by body movements and respiration), low-pass filters (that remove the high-frequency noise such as electrical interference or muscle noise), and so forth. In an embodiment, the amplified and filtered ECG signal 134 may be available at an output pin of the AD8232-module-based sensor 110. In an embodiment, the AD8232-module-based sensor 110 is configured to display the ECG signal 134 on a serial monitor (i.e., Arduino Integrated Development Environment (IDE)). For example, the Arduino may read the ECG signal 134 from the AD8232-module-based sensor 110 through an analog input pin of the Arduino. Then, the ECG signal 134 may be transmitted to the serial monitor for visualization. In an exemplary embodiment, the Arduino may be connected to the AD8232-module-based sensor 110 by connecting a signal output pin of the AD8232-module-based sensor 110 to the analog input pin of the Arduino, a ground pin of the AD8232-module-based sensor 110 to a ground pin of the Arduino and a power supply pin of the AD8232-module-based sensor 110 to a power supply pin of the Arduino. In another embodiment, the AD8232-module-based sensor 110 is configured to store the ECG signal 134 in a database (not shown) for further analysis. In yet another embodiment, the AD8232-module-based sensor 110 is configured to transmit the ECG signal 134 wirelessly to a remote server for real-time monitoring.

The IoT-based signal transmission circuitry 104 is configured to facilitate seamless communication between the ECG signal acquisition circuitry 102 and the Fog-network-based signal processing circuitry 106. The IoT-based signal transmission circuitry 104 is configured to use IoT communication to transmit the ECG signal 134 to the Fog-network-based signal processing circuitry 106. The IoT-based signal transmission circuitry 104 includes an Arduino-Nano-3-module-based controller 114 and a wireless communication component such as an ESP8266 wireless communication component 116 (manufactured by Espressif Inc, Room9A Jingxing Building, Changyong Road, Long Hua Xin Qu District, Shenzhen, China).

The Arduino-Nano-3-module-based controller 114 is a central controller that integrates and connects the AD8232-module-based sensor 110 and the ESP8266 wireless communication component 116 to enable reception, transmission and processing of the ECG signal 134. In an embodiment, an output pin of the AD8232-module-based sensor 110 may be connected to an analog pin (A1) of the Arduino-Nano-3-module-based controller 114, and the Arduino-Nano-3-module-based controller 114 may read the analog ECG signal 134 from the analog pin (A1).

The Arduino-Nano-3-module-based controller 114 is configured to process the ECG signal 134 to make the ECG signal 134 compatible with the IoT transmission. In an embodiment, the Arduino-Nano-3-module-based controller 114 may be integrated with a microcontroller (e.g., ATmega328) to process analog voltage readings corresponding to the ECG signal 134 captured by the AD8232-module-based sensor 110. In an embodiment, the Arduino-Nano-3-module-based controller 114 is configured to process the ECG signal 134 by performing various tasks such as, but not limited to, analog-to-digital conversion, filtering, amplification, and so forth. In an exemplary embodiment, the ADC may be performed by using an Analog-To-Digital Converter (ADC) that converts analog voltage values into digital values. In another exemplary embodiment, the filtering may be performed by using smoothing algorithms that reduce any residual noise from the ECG signal 134.

The Arduino-Nano-3-module-based controller 114 may be configured to transmit the processed ECG signal 134 to the ESP8266 wireless communication component 116 using a serial communication protocol through digital pins 2 and 3. The serial communication protocol may be a Universal Asynchronous Receiver/Transmitter (USART), a Serial Peripheral Interface (SPI), a serial-over-Bluetooth, and so forth. Also, the digital pins 2 and 3 may refer to physical pins on a board of the Arduino-Nano-3-module-based controller 114, and configured for digital input or output operations. For example, the digital pins 2 and 3 on the Arduino-Nano-3-module-based controller 114 may be used to communicate with the ESP8266 wireless communication component 116.

In an embodiment, the ESP8266 wireless communication component 116 may establish a wireless connection to an access point (i.e., Wireless-Fidelity (Wi-Fi) network) using a wireless channel. The wireless channel may be, but not limited to, Wi-Fi, Zigbee, cellular networks, Long Range (LoRa), Radio Frequency (RF), and so forth. Upon establishing the connection, the ESP8266 wireless communication component 116 transmits the ECG signal 134 wirelessly to the Fog-network-based signal processing circuitry 106 using IoT communication protocols. The IoT communication protocols may be, but not limited to, Message Queuing Telemetry Transport (MQTT), Narrowband IoT (NB-IoT), Zigbee, and so forth.

The Fog-network-based signal processing circuitry 106 is configured to receive the ECG signal 134 from the ESP8266 wireless communication component 116. The Fog-network-based signal processing circuitry 106 is configured to use a Fog network 118 to apply a modified Pan and Tompkins algorithm 138 (as shown in FIG. 1B) to extract a plurality of ECG features (hereinafter referred to as the ECG features) from the ECG signal 134 in a frequency domain and time domain. The Fog network 118 refers to a distributed computing infrastructure that extends cloud computing capabilities closer to the edge of a network, where data is generated (e.g., IoT devices). As used herein, the term “frequency domain” refers to a signal in terms of its frequency components rather than time and the term “time domain” represents the ECG signal 134 as it changes over time. The modified Pan and Tompkins algorithm 138 includes an improved filtering stage, a negative peak handling stage, an improved dynamic threshold mechanism, an improved differentiation stage and an improved feature extraction stage.

The improved filtering stage cleans up the ECG signal 134, reduces the noise and minimizes false PVC detections. The negative peak handling stage detects both positive and negative peaks, which enhances an accuracy of detecting the QRS complexes when dealing with abnormal heartbeats. The improved dynamic thresholding mechanism adjusts a threshold level (i.e., used to differentiate specific ECG features in the ECG signal 134) according to variations in an amplitude of the signals and noise levels. The dynamic thresholding mechanism reduces the false PVC detections, ensuring that the modified Pan and Tompkins algorithm 138 accurately detects the QRS complexes, even in challenging conditions (e.g., noisy signals or varying characteristics of the ECG signal 134). The improved differentiation stage derives gradient (slope) values accurately using a transfer function, which results in precise detection of onset and offset of the QRS complexes, which is essential for accurate analysis of the ECG signal 134.

The improved feature extraction stage extracts the ECG features that may be essential for detecting the PVC arrhythmia. In an embodiment, the extracted ECG features include QRS complex durations, R-peaks, R-R intervals, an average of the R-R intervals, an average deviation of the R-R intervals, standard deviation of successive difference and so forth. As used herein, the term “QRS complex duration” refers to a length of time required for the QRS complex to occur in the ECG signal 134. In an embodiment, the QRS complex often exhibits variable durations in the ECG signal 134 compared to the typical 80-120 milliseconds that may be seen in healthy individuals. To address this variability, conditions may be established to classify the QRS complexes based on a block width, which represents the duration of waveform segment under analysis. If the block width meets or exceeds a predefined threshold corresponding to an expected QRS duration, then the waveform segment is classified as the QRS complex. This ensures accurate capture of both arrhythmia pulses and average healthy rhythms. When the block width is shorter than the predefined threshold, then the system 100 further classifies the ECG signal 134 into the noise, P-waves, or T-waves.

Also, as used herein, the term “R-R intervals” refers to a time duration between the R-peak of a previous cardiac cycle (i.e., previous R-interval) and the R-peak of a successive cardiac cycle (i.e., subsequent R-interval) in the ECG signal 134, representing one complete cardiac cycle.

Also, as used herein, the term “previous R-interval” refers to a time between a current heartbeat and a previous heartbeat. As used herein, the term “subsequent R-R interval” refers to a distance between two consecutive heartbeats, helping to analyze a pattern of heartbeats, including irregular rhythm detection. As used herein, the term “standard deviation of successive difference” refers to an index that quantifies a variation in the R-R intervals between successive heartbeats. The standard deviation of successive difference determines how regular or irregular the rhythm is, especially for detecting arrhythmias. As used herein, the term “average of the R-R intervals” refers to an average of multiple R-R intervals over a series of consecutive heartbeats. The average of the R-R intervals provides a record of healthy cardiac rhythm and detects abnormalities signalizing the arrhythmias. Also, as used herein, the term “average deviation of the R-R intervals” refers to an average of absolute differences between each individual R-R interval and the average R-R interval over a given period. The rate of change of the R-R interval is derived from the average deviation of the R-R interval.

The Fog-network-based signal processing circuitry 106 is configured to determine whether the patient has PVC arrhythmias based on the extracted ECG features. In an embodiment, the Fog-network-based signal processing circuitry 106 is configured to input the extracted ECG features into Cyclic Echo State Network (CESN) 140 (as shown in FIG. 1B) trained for classifying PVC heartbeats and normal heartbeats. In an embodiment, working and training of the CESN 140 may be explained in conjunction with FIGS. 1B and 1D. The Fog-network-based signal processing circuitry 106 is configured to use the Fog network 118 to provide an alert to the patient and a healthcare team upon determining that the patient has the PVC arrhythmias. In an exemplary embodiment, the alert may be an audio alert, a visual alert, a digital alert, and so forth. In an embodiment, the Fog-network-based signal processing circuitry 106 may be configured to transmit the alert through the IoMT-connected devices to the healthcare team. In an embodiment, the alert may be transmitted in form of Short Message Service (SMS), emails, or through a healthcare management platform.

FIG. 1B illustrates an exemplary system workflow architecture 120, according to certain embodiments. The system workflow architecture 120 illustrates a workflow and an interaction between components of the system 100. The components include a data acquisition unit 122, a preprocessing unit 124, a classification unit 126 and a performance evaluation unit 128.

The data acquisition unit 122 is configured to collect and utilize datasets from different sources, such as a real-time ECG monitoring module 130 and an open-access arrhythmia database 132 (e.g., PhysioNet). In an embodiment, the real-time ECG monitoring module 130 may be configured to capture real-time recordings of the ECG signal 134. The real-time recordings of the ECG signal 134 may be collected from a predefined (i.e., 30) number of patients, each with a recording duration of 20-minutes, for example, from a cardiology ward at ABC hospital. In an embodiment, the real-time recordings of the ECG signal 134 may be raw ECG signal captured through the electrodes 112 (as shown in FIG. 1A) and processed by the AD8232-module-based sensor 110 (as shown in FIG. 1A) of the ECG signal acquisition circuitry 102 (as shown in FIG. 1A).

Further, the data acquisition unit 122 is configured to receive benchmark dataset 136 (i.e., XYZ Hospital arrhythmia dataset) from the open-access arrhythmia database 132. The benchmark dataset 136 serves as a reference point for assessing the accuracy, efficiency and robustness of the system 100 under controlled conditions. In an exemplary embodiment, the benchmark dataset 136 may include pre-classified ECG signals with annotations for different types of heartbeats, including the PVC heartbeats (represent extra heartbeats originating in the ventricles, which can disrupt normal heart rhythm), the normal heartbeats (represent healthy heart activity), supraventricular ectopic heartbeats (represent the abnormal heartbeats originating above the ventricles), unknown heartbeats (represent heartbeats that do not fall into the above categories and are harder to classify), and ventricular and normal heartbeats. The benchmark dataset 136 provides a standardized reference for testing the accuracy of the system 100 in detecting the R-peaks, the QRS complexes, and the PVC arrhythmias.

The data acquisition unit 122 integrates the real-time recordings of the ECG signal 134 and the benchmark dataset 136 to develop and validate the system 100. By integrating the real-time recordings of the ECG signal 134 and the benchmark dataset 136, the data acquisition unit 122 ensures that the system 100 benefits from real-world applicability and standardized validation. The real-time recordings of the ECG signal 134 provide dynamic, patient-specific signals, while the benchmark dataset 136 offers a controlled environment for performance evaluation of the system 100. The combination of the real-time recordings of the ECG signal 134 and the benchmark dataset 136 ensures that the system 100 is robust, accurate, and capable of detecting a wide range of heartbeats, including the normal heartbeats and the PVC heartbeats.

The preprocessing unit 124 is communicatively coupled with the data acquisition unit 122. The preprocessing unit 124 is configured to receive the captured real-time recordings of the ECG signal 134 and the benchmark dataset 136 from the data acquisition unit 122. The preprocessing unit 124 is configured to process the real-time recordings of the ECG signal 134 using the modified Pan and Tompkins algorithm 138. The preprocessing unit 124 is configured to process the real-time recordings of the ECG signal 134 for cleaning and preparing the real-time recordings of the ECG signal 134 for analysis and classification. For instance, the preprocessing unit 124 may be configured to filter the noise, such as the baseline drifting, the power line interference and motion artifacts from the real-time recordings of the ECG signal 134. Further, the preprocessing unit 124 may be configured to apply the modified Pan and Tompkins algorithm 138 for QRS complex detection and R-peak identification. The modified Pan and Tompkins algorithm 138 may use gradient-based methods to enhance steep slopes of the QRS complex. For example, the Pan and Tompkins algorithm 138 may be modified to address clinical requirements, such as improving the detection accuracy of the QRS complexes, handling inter-patient fluctuations (i.e., variations in the ECG signal 134 due to differences between the patients), handling negative polarity values, correctly identifying borders of the QRS complexes, as the borders of the QRS complexes are essential for distinguishing between the normal heartbeats and the PVC heartbeats. The modified Pan and Tompkins algorithm 138 enables accurate estimation of R-peak locations, facilitating extraction of the pertinent ECG features from the real-time recordings of the ECG signal 134, which are subsequently transmitted to the classification unit 126.

In an embodiment, the benchmark dataset 136 ensures that the modified Pan and Tompkins algorithm 138 performs accurately when dealing with known and annotated ECG patterns. For example, the annotated ECG patterns in the benchmark dataset 136, such as the XYZ hospital arrhythmia dataset, include labels like normal sinus rhythm, PVC, atrial fibrillation (AFIB) and so forth. The labels may serve as ground truth for validating the ability of the system 100 to detect and classify the heartbeats accurately.

In an exemplary embodiment, the system 100 is tested against the benchmark dataset 136 to evaluate how well the modified Pan and Tompkins algorithm 138 performs in detecting the heartbeats, the R-peaks, the QRS complexes, and various arrhythmias, including the PVC heartbeats. For example, the system 100 may process the real-time recordings of the ECG signal 134 and detect a series of PVC heartbeats. The detected series of PVC heartbeats may be compared with corresponding PVC annotations in the benchmark dataset 136. If the system 100 identifies 95 out of 100 PVC heartbeats correctly, then the detection accuracy of the PVC heartbeats is quantified as 95%.

In an embodiment, the benchmark dataset 136 serves as the controlled environment for validating feature extraction, heartbeat detection and classification capabilities of the system 100. By comparing real-time processing results, such as timing and morphology of the R-peaks and the QRS complexes, with the benchmark dataset 136, inconsistencies may be identified. For example, if the system 100 consistently overestimates R-peak locations by 5 milliseconds compared to the benchmark dataset 136, then the adjustments are made to the Pan and Tompkins algorithm 138 to enhance the performance of the system 100.

The classification unit 126 is communicatively coupled to the preprocessing unit 124. The classification unit 126 is configured to receive the extracted ECG features from the preprocessing unit 124. The classification unit 126 is configured to train and deploy a classifier, such as the CESN 140 for generating an output 142, for example, classifying the PVC heartbeats and the normal heartbeats 142. In an embodiment, the CESN 140 may be trained using a supervised learning technique. The training of the CESN 140 includes providing the CESN 140 with labeled data, where the ECG signal 134 is annotated with known labels, such as “PVC” or “normal”. The supervised learning technique allows the CESN 140 to learn to differentiate between the normal heartbeats and the PVC heartbeats by mapping the extracted ECG features to the corresponding known labels. The CESN 140 processes the ECG features and generates a binary classification output. In an embodiment, the CESN 140 assigns a binary value to each heartbeat, such as a value of “O” representing the normal heartbeat and a value of “1” representing the PVC heartbeat.

The performance evaluation unit 128 is communicatively coupled to the classification unit 126. The performance evaluation unit 128 is configured to evaluate the performance of the CESN 140, in classifying the PVC heartbeats and the normal heartbeats 142. The performance evaluation unit 128 is configured to evaluate the performance of the CESN 140 by using a tenfold cross-validation technique. The tenfold cross-validation technique splits an available ECG dataset into multiple subsets (folds) to ensure that the CESN 140 is evaluated on different data portions, reducing bias and variance in the performance evaluation. For example, the dataset is split into 10 equal folds. In each iteration, one fold is used as a test set, and the remaining 9 folds are used for training. The process is repeated 10 times, with each fold being used once as the test set. This ensures that every instance in the dataset is used for both training and testing. The results of all 10 iterations are averaged to get a robust estimate of the performance of the CESN 140.

The tenfold cross-validation technique ensures that the CESN 140 generalizes across different ECG datasets and detects critical arrhythmias (like PVC) reliably. The performance evaluation unit 128 is configured to evaluate the output 142 of the CESN 140 by using standard performance metrics such as, but not limited to, classification accuracy (measures overall proportion of correctly classified heartbeats), sensitivity (evaluate classifier's ability in correctly classifying the PVC heartbeats), specificity (evaluate classifier's effectiveness in correctly classifying the normal heartbeats), precision (represents proportion of correctly identified PVC heartbeats among all predicted PVC heartbeats), F1 score (combines sensitivity and precision into a single metric to provide balanced measure of classifier's performance), Area Under the Curve (AUC) (evaluates classifier's ability to distinguish between the normal heartbeats and the PVC heartbeats over varying thresholds), G-mean (calculates a geometric mean of sensitivity and specificity to provide an overall measure of the classifier's performance), error rate (represents proportion of incorrect predictions by the classifier), and so forth. In an embodiment, the performance metrics may be computed based on guidelines specified by Association for the Advancement of Medical Instrumentation (AAMI) criteria. By adhering to the AAMI criteria, the performance evaluation unit 128 ensures that the system 100 meets industry standards for medical device performance, confirming its effectiveness in real-world medical applications, and is ready for deployment as a reliable ECG monitoring system for detecting the PVC arrhythmias.

FIG. 1C illustrates a block diagram showing stages of processing the ECG signal 134 performed by the Fog network 118 using the modified Pan and Tompkins algorithm 138, according to certain embodiments. In an embodiment, the ECG signal 134 may include various noises such as the baseline drifting, the power line interference, unstable electrode-skin contact, and so forth. As used herein, the term “baseline drifting” refers to slow and low-frequency variations in a baseline level of the ECG signal 134, caused by external or physiological factors such as patient movement, breathing (respiratory variations), changes in electrode-skin contact and so forth. Also, as used herein, the term “power line interference” refers to unwanted signals in the ECG signal 134 that are caused by electromagnetic noise from electrical power sources. The power line interference occurs when power lines emit electromagnetic fields that couple into the ECG signal 134, distorting ECG waveform.

The noises may affect the ECG signal 134 and complicate rhythm analysis, leading to inaccurate classification. To remove the noises from the ECG signal 134, the Fog network 118 (as shown in FIG. 1A) is configured to perform signal preprocessing on the ECG signal 134 to obtain the preprocessed ECG signal 134. The signal preprocessing includes signal normalization and signal filtering. The signal normalization may be performed by using a min-max normalization technique that may be applied to rescale values of the ECG signal 134 within a specific range to obtain the normalized ECG signal 134. In an embodiment, a magnitude of the normalized ECG signal 134 falls within a predefined ECG signal magnitude range. In a preferred embodiment, the predefined ECG signal magnitude range is [0,1] or [−1,1]. In an embodiment, the signal normalization may be performed by using equation (1):

$$normalization = \frac{( x - min ( x ) )}{( ( max ( x ) - min ( x ) )} \qquad ( 1 )$$

where x is an original (raw) ECG signal 134 and min(x) and max(x) are minimum and maximum values of the ECG signal 134.

The signal filtering includes a median filter 144 and a Finite Impulse Response (FIR) filter 146. The median filter (non-linear digital filtering) 144 removes the baseline drifting from the normalized ECG signal 134. In an exemplary embodiment, the median filter 144 removes the baseline drifting by replacing each data point in the normalized ECG signal 134 with a median value of neighboring data points within a specified window. Since the baseline drifting affects the ECG signal 134 in a slow and gradual manner, the median filter 144 is effective because it preserves sharp features (such as the QRS complex) of the ECG signal 134 while smoothing out low-frequency signal components, ensuring that only relevant high-frequency signal components are retained for analysis.

The FIR filter 146 acts as a low-pass filter and removes the high-frequency noise, such as the power line interference from the normalized ECG signal 134. In an exemplary embodiment, the FIR filter 146 may remove the power line interference in the normalized ECG signal 134 by using a notch filter design that specifically targets and attenuates the power line interference at a known frequency of 50 Hertz (Hz) or 60 Hz, which is a frequency of a power line noise. The FIR filter 146 applies a set of predefined coefficients to the normalized ECG signal 134. The set of predefined coefficients is designed so that the FIR filter 146 responds only to the noise at a specific frequency without affecting the rest of the normalized ECG signal 134. For example, in the notch filter, the set of predefined coefficients may be [1, −2, 1] that create a notch in a frequency response at a desired frequency, which filters out the power line interference. By filtering out the power line interference while maintaining the integrity of the normalized ECG signal 134, the FIR filter 146 ensures that the ECG features like the QRS complex and the R-peak are not distorted, enabling PVC classification and diagnosis.

The combination of the FIR filter 146 and the median filter 144 helps isolate valuable features (like the QRS complex) of the ECG signal 134 by removing unwanted noise and preserving the integrity of the ECG signal 134.

Further, the Fog network 118 is configured to use a differentiation circuitry 148 to extract gradient data from the preprocessed ECG signal 134. The gradient data refers to a rate of change, or slope, of the preprocessed ECG signal 134 at a specific time instance. As used herein, the term “specific time instance” refers to a particular moment corresponding to a discrete sample point in the preprocessed ECG signal 134, representing the electrical activity of the heart as measured at that particular time. The differentiation circuitry 148 applies a mathematical derivative operation to the preprocessed ECG signal 134. The mathematical derivative operation calculates a difference in signal amplitude between consecutive time points in the preprocessed ECG signal 134 to estimate how rapidly the signal amplitude changes at specific intervals. The mathematical derivative operation generates a new signal representing the rate of change (gradient data) in the preprocessed ECG signal 134 over time. In other words, the new signal is generated by applying a transfer function that calculates the difference between consecutive sample points in the preprocessed ECG signal 134, providing a first-order approximation of derivative of the preprocessed ECG signal 134. The transfer function acts as a tool to compute the rate of change at each specific time instance.

For instance, when the preprocessed ECG signal 134 is flat (e.g., between the heartbeats), the gradient data is close to zero, indicating minimal change in the preprocessed ECG signal 134. As the preprocessed ECG signal 134 approaches the QRS complex, the gradient data increases significantly because the preprocessed ECG signal 134 undergoes rapid changes. At the peak of the QRS complex, the gradient data reaches its maximum, indicating a sharpest rate of change.

The differentiation circuitry 148 analyzes the gradient data to locate the borders of the QRS complex, which corresponds to its onset and offset. The onset of the QRS complex is identified as a point where the gradient data rises sharply from a low value, indicating a beginning of a steep upward transition in the preprocessed ECG signal 134. The offset of the QRS complex is determined as a point where the gradient drops back to near zero levels, indicating an end of the rapid signal change associated with the QRS complex.

The differentiation circuitry 148 is further configured to derive high slope values by calculating a plurality of derivative points based on the transfer function. Each derivative point corresponds to the rate of change of the preprocessed ECG signal 134 at the specific time interval. Among the generated derivative points, the derivative point with the highest magnitudes is identified as the high slope value, indicating the steepest transition in the preprocessed ECG signal 134. The high slope values are crucial for accurately identifying the borders of the QRS complex, as the high slope values correspond to the most prominent changes in the signal amplitude.

The Fog network 118 is further configured to use the squaring circuitry 150 to perform non-linear amplification on the extracted gradient data. In an exemplary embodiment, the squaring circuitry 150 performs the non-linear amplification on the extracted gradient data (or derivative data) by amplifying higher frequency components of the derivative of the preprocessed ECG signal 134. The amplification of the higher frequency components is achieved by squaring the derivative values of the preprocessed ECG signal 134. For example, a squaring operation takes the gradient data (or derivative values) of the preprocessed ECG signal 134 and squares the derivative values of the preprocessed ECG signal 134.

By squaring the derivative values of the preprocessed ECG signal 134, the squaring circuitry 150 amplifies the steepest portions of the preprocessed ECG signal 134, which correspond to the QRS complex. The squaring operation enhances regions of the preprocessed ECG signal 134 with higher derivatives (higher slopes), making the regions disproportionately larger than regions with lower slopes. As a result, the squaring circuitry 150 emphasizes sharp transitions that are critical for the QRS complex detection. Since the QRS complex has a sharp slope (or gradient), the squaring circuitry 150 increases the magnitude of the QRS complex by performing the squaring operation. The squaring circuitry 150 also removes any negative values from the gradient data, ensuring all values are positive. After squaring, the preprocessed ECG signal 134 is amplified such that the important features (i.e., the sharp slopes of the QRS complex) are more prominent, improving the accuracy of R-peak detection and QRS identification.

The Fog network 118 is further configured to use moving integrating circuitry 152 to extract the ECG features in the frequency domain and time domain. The moving integrating circuitry 152 includes two event-related moving averages such as a first moving average and a second moving average, to enhance the accuracy of extraction of the ECG features.

The first moving average (i.e., QRS moving average) is a signal processing technique applied to the raw ECG signal 134 to smooth out the noise and emphasize the ECG features, such as the QRS complex. The first moving average smooths the ECG signal 134 by calculating an average value of the ECG signal 134 over a first sliding window of a fixed length. As used herein, the term “first sliding window” is a segment of data that slides over the ECG signal 134 one sample at a time. For example, in the ECG signal 134, the first moving average may use the first sliding window for a duration of 150-200 milliseconds (ms). At each step, the first moving average computes the average value of the ECG signal 134 within that window. Further, the window shifts by one sample, and the process repeats for the entire ECG signal 134. The average of the ECG signal 134 indicates areas of the ECG signal 134 where the QRS complex is present, making the QRS complex easier to detect. In an exemplary embodiment, the first moving average smooths down multiple peaks that may occur within the QRS complex duration in the ECG signal 134 due to the noise, the baseline drifting and so forth. The multiple peaks may create difficulties in identifying true QRS complex. Therefore, the first moving average smooths down the multiple peaks by averaging values within the first sliding window.

The second moving average (i.e., one-beat moving average) is applied to an output of the first moving average to refine the ECG signal 134 further and emphasize on individual heartbeats. The second moving average operates by calculating an average of the smoothed ECG signal 134 over a second sliding window (e.g., 200-300 milliseconds), which is slightly larger than the first sliding window. In an exemplary embodiment, the second moving average takes the smoothed ECG signal 134 from the first moving average and applies the second sliding window, which computes an average signal value within a defined window size as it moves sample by sample across the ECG signal 134. This additional smoothing step enhances the QRS complex by suppressing smaller fluctuations and artifacts, making it easier to detect the ECG features while ensuring the integrity of the overall ECG signal 134. The second moving average provides a cleaner version of the ECG signal 134, which is critical for detecting actual R-peaks in the QRS complex. The second moving average accentuates the QRS complex and provides an adaptive threshold for the first moving average, to improve the precision of the R-peak detection.

Further, the ECG signal 134 that is processed through the first moving average and the second moving average is segmented into blocks of interest, which represent potential QRS complexes. In an embodiment, each block corresponds to a specific time interval and may be evaluated for cardiac events. In an embodiment, each block is analyzed using an event-related threshold to determine the classification of each block. In an exemplary embodiment, if the amplitude and other features of the block exceed the adaptive threshold, then the corresponding block is classified as containing the R-peak and the block is a part of the QRS complex. In another embodiment, if the amplitude and other features of the block are less than the adaptive threshold, then the block is classified as the noise, the P-wave or the T-wave.

The decision-making process 154 includes interpreting the processed ECG signal 134 to classify detected events, such as distinguishing between the normal heartbeats and the PVC heartbeats. The decision-making process 154 relies on the extracted ECG features, such as the R-R intervals, the QRS complex durations, and so forth, to make accurate classification. In an embodiment, using the extracted ECG features, the decision-making process 154 applies dynamic thresholds or static thresholds to classify the events. For example, if the R-R interval of the processed ECG signal 134 is shorter than a normal range, the decision-making process 154 may indicate the PVC heartbeat. Similarly, if the QRS complex duration of the processed ECG signal 134 exceeds a predefined threshold, then the decision-making process 154 may indicate ventricular arrhythmia.

Suppose the ECG signal 134 is processed and various ECG features such as the R-R interval, the QRS complex duration, the R-peaks, the average of the R-R intervals and the average deviation of the R-R intervals are extracted. In this example, the R-R interval is measured at 600 ms (where a normal range is 700-1000 ms), the QRS complex duration is 150 ms (with a normal range being 80-120 ms) and the R-peaks detected at regular intervals. The average of the R-R interval is 650 ms (lower than normal, indicating irregular rhythm) and the average derivation of the R-R intervals is 50 ms (indicating significant variability). Based on the analysis, the decision-making process 154 detects that a short R-R interval and high variability in the average of the R-R interval indicate the irregular rhythm. The prolonged QRS complex duration indicates that the heartbeats likely originate from the ventricles, and irregular R-peak detection supports the possibility of premature heartbeat. As a result, the system 100 classifies this pattern as a PVC event and alerts the healthcare team to take immediate action.

The combining paths 156 merge outputs from multiple signal processing stages to prepare a complete set of ECG features for the classification of the processed ECG signal 134. In an exemplary embodiment, the combining paths 156 may merge the outputs from the signal filtering (i.e., the median filter 144 and the FIR filter 146), the differentiation circuitry 148 and the squaring circuitry 150 to provide the set of ECG features. For example, the signal filtering removes the unwanted noise, such as the baseline drifting or the powerline interference, from the ECG signal 134, to provide essential features such as the QRS complex. The differentiation circuitry 148 provides the borders of the QRS complex, which are crucial for accurately detecting the heartbeats. The squaring circuitry 150 performs the non-linear amplification on the preprocessed ECG signal 134 by squaring the differentiated ECG signal 134. The squaring circuitry 150 emphasizes the high-frequency components associated with the QRS complex, making detecting the R-peaks and other relevant features of the ECG signal 134 easier. By merging the outputs from the multiple signal processing stages, the combining paths 156 ensures that a final representation of the ECG signal 134 is robust, clean and suitable for further classification.

In an embodiment, the QRS complex duration and the R-peak are primary ECG features for identifying the PVC by using the modified Pan and Tompkins algorithm 138 and the CESN 140. However, additional features such as the R-R interval, the previous R-R interval, and the subsequent R-R interval may also be extracted to aid in classification.

In an embodiment, the extraction of R-peak locations and computation of the R-R intervals are crucial steps in ECG signal 134 analysis, particularly for identifying the PVC. In an embodiment, the R-peak locations are extracted from ECG readings by identifying the R-peaks representing the highest points in the QRS complex within the preprocessed ECG signal 134. Once the R-peak locations are identified, the subsequent R-R interval is calculated by determining a time difference between consecutive R-peaks, using equation (2)

$$Subsequent R - R interval = {R}_{p e a k} ( j + 1 ) - {R}_{p e a k} ( j ) \qquad ( 2 )$$

where Rₚₑₐₖ (j) is the location of the current R-peak and Rₚₑₐₖ (j+1) is the location of the subsequent R-peak.

Similarly, the previous R-R interval is calculated using equation (3) to compare the current and previous R-R intervals, which helps distinguish the PVC heartbeats from the normal heartbeats.

$$Previous R - R interval = {R}_{p e a k} ( j ) - {R}_{p e a k} ( j - 1 ) \qquad ( 3 )$$

where Rₚₑₐₖ (j−1) is the location of the previous R-peak.

In cases where the R-peaks are missed or misidentified due to the noise, low amplitudes, or distinct PVC structures, an algorithm rechecks the ECG signal 134 to ensure accurate detection, including scanning for inverse R-peaks. As used herein, the term “inverse R-peaks” refers to negative deflections in the ECG signal 134 that resemble the R-peak, but appear inverted due to specific lead placement or heart's electrical axis.

The Adaptive Decision-Making Threshold (ADMT) 158 refines the ECG signal 134 analysis by filtering out irrelevant data (i.e., parts of the ECG signal 134 that do not contribute to PVC detection) and minimizing the impact of the noise. The ADMT 158 ensures that only relevant features, such as the QRS complexes, which are indicative of the PVC, are considered for analysis. The irrelevant data may be the baseline drifting, the T-waves, noise-induced artifacts, and so forth.

The ADMT 158 dynamically adjusts a threshold for detecting significant cardiac events. The adjustment is based on real-time features of the ECG signal 134, such as the amplitude, noise levels, and frequency. This adaptive behavior enables the system 100 to maintain accuracy despite variations in signal quality, which may occur due to factors like patient movement, noise interference, different heart conditions and so forth. For example, if the ECG signal 134 has low amplitudes or is affected by the high-frequency noise, the ADMT 158 may lower the threshold to avoid false positives or negatives. On other hand, if the ECG signal 134 is cleaner, then the ADMT 158 may raise the threshold to accurately detect the QRS complexes without considering the noise as a valid event. The ADMT 158 is useful in scenarios like inter-patient variability where the ADMT 158 may effectively adjust to differences in the ECG signal 134 across different individuals, ensuring the detection of the real cardiac events is precise, regardless of external conditions.

FIG. 1D illustrates an architecture 160 of the CESN 140, according to certain embodiments. The architecture 160 of the CESN 140 includes an input layer 162, a reservoir (fixed Recurrent Neural Network (RNN) pool) 164, and an output layer 166. The architecture 160 is designed to process the extracted ECG features (i.e., temporal features extracted from the raw ECG signal 134), with high efficiency and accuracy.

The input layer 162 is configured to deliver the ECG features 134 to the reservoir 164 through weighted connections. The weighted connections are characterized by numerical values called weights assigned to links between the input layer 162 and the reservoir 164. The weights determine how the ECG features are distributed and scaled when the ECG features enter the reservoir 164. In an embodiment, the weights may be fixed and randomly initialized during setup of the system 100 and not altered during training of the reservoir 164. In another embodiment, the weights may be scaled to control the strength of the ECG features that are fed into the reservoir 164. In yet another embodiment, the weighted connections between the input layer 162 and the reservoir 164 are sparse, indicating that only a subset of neurons of the reservoir 164 receive the ECG features from the input layer 162.

The reservoir 164 may be designed to process and transform the ECG features into a high-dimensional state space, capturing the temporal and dynamic patterns within the ECG features. As used herein, the term “high-dimensional state space” refers to a mathematical representation where the points are expressed in a space with a large number of dimensions. In an exemplary embodiment, the transformation may include normalization (i.e., adjusting the amplitude of the ECG features to fall within a defined range (e.g., 0 to 1 or −1 to 1)), scaling (i.e., multiplying the normalized ECG features by a scaling factor to adjust range of the ECG features before feeding the ECG features into the reservoir 164), and so forth. By transforming a low-dimensional input (i.e., the ECG features) into the suitable format, the input layer 162 ensures that the ECG features are mapped into the high-dimensional state space of the reservoir 164.

The reservoir 164 may include a network of interconnected neurons with fixed, randomly initialized weights. The weights define how the ECG features propagate through the reservoir 164 and interact with its states.

For example, the reservoir 164 processes incoming time-series data (i.e., the ECG features) to identify patterns associated with the normal heartbeats and the PVC heartbeats. For instance, as the ECG features enter the reservoir 164, the amplitude and the temporal features (e.g., P-waves, QRS complexes, T-waves) are mapped to the high-dimensional space where each neuron captures a unique aspect of the ECG signal 134. In an embodiment, a cyclic structure of the reservoir 164, formed by interconnections between the neurons, ensures that the ECG features flow through recurrent pathways, enabling the system 100 to retain historical information and model temporal dependencies. The cyclic and recurrent structure allows the reservoir 164 to preserve temporal patterns like heart rate variability or arrhythmia-related irregularities.

The output layer 166 is a final stage of the architecture 160 and may be designed to transform the high-dimensional state space produced by the reservoir 164 into the output 142, such as classifying the ECG signal 134 into the PVC heartbeats and the normal heartbeats. The output layer 166 combines (i.e. linear combination) the states of the reservoir 164 using trainable weights. The linear combination includes summing up weighted combinations from all the states in the reservoir 164. Each state of the reservoir 164 represents the dynamic and temporal patterns captured from the ECG features. The linear combination enables the output layer 166 to integrate the various patterns captured in the reservoir 164 into a single output signal.

For example, when analyzing the ECG features, the states in the reservoir 164 may represent patterns such as the amplitude and the timing of different segments of the electrical activity of the heart, like the QRS complex or the P-wave. The linear combination of the states may enable the output layer 166 to make a decision based on the ECG features such as classifying the heartbeat as the normal heartbeat or the PVC heartbeat.

In an embodiment, the connections between the states of the reservoir 164 and the output layer 166 are governed by the trainable weights. The weights are adjusted during a training process to map the states of the reservoir 164 to a desired output. The training process includes adjusting the weights in a way that minimizes error between the output 142 and a true label, allowing the output layer 166 to learn how to make accurate predictions.

In an embodiment, the output layer 166 is the only layer of the CESN 140 where the weights are updated during the training process. The output layer 166 is trained by using the supervised learning technique. The supervised learning technique is a process where a model is trained on labeled data, which means that each input sample (e.g., ECG signal 134) is paired with a known correct output (i.e., a label indicating whether the heartbeat is normal or PVC). During the training process, the weights of the output layer 166 are adjusted to minimize classification errors which is performed by using a loss function. By adjusting the weights based on the labeled data, the output layer 166 becomes more accurate at classifying the ECG signal 134.

In an embodiment, the output layer 166 may include a feedback layer that improves a learning process of the CESN 140. The feedback layer provides iterative feedback that allows the CESN 140 to refine classification abilities. During the training process, the feedback may be used to adjust a gain or weight of feedback connections, which helps the CESN 140 to learn from its previous outputs and make more accurate predictions. As used herein, the “gain of feedback” refers to intensity of a correction signal sent back to the CESN 140. A higher feedback gain allows the CESN 140 to make more adjustments to its weights based on previous errors and a lower feedback gain results in smaller adjustments.

Further, in an embodiment, the CESN 140 may use a window-based segmentation to handle variations between the different patients, as the ECG features from the different patients often exhibit variability due to individual heart conditions. The window-based segmentation is a technique used to divide the extracted ECG features into smaller and manageable windows. The window-based segmentation ensures that each window contains relevant ECG features like the QRS complex, the P-waves, and the T-waves (important for heartbeat identification), thus reducing the variability between the individual patients.

For example, in a typical scenario, the ECG features are divided into windows of a fixed length (i.e., 200 samples long) of 0.52 seconds, surrounding each R-peak (i.e., the R-peak is placed near the center of window) of the detected heartbeat. For instance, for the patient with a heart rate of 75 beats per minute, one window may represent a fraction of a second surrounding the R-peak of each heartbeat. By consistently segmenting the ECG features, even if the patient has irregular heart rhythms, the relevant ECG features for the classification may always be present in each window. By segmenting the ECG features into fixed-size windows, each window may be processed independently, ensuring that the CESN 140 receives consistently structured input data regardless of these variations.

The extracted ECG features may contain information associated with temporal characteristics (timing-related) and morphological characteristics (i.e., the shape of P-wave, QRS complex, and T-wave, which can vary based on heart conditions) of each heartbeat. The extracted ECG features from each window may be fed into the CESN 140 as input. In this way, each window has the same structure so that for any patient, the data (i.e., the ECG features) being fed to the CESN 140 is structurally similar even if the morphology of heartbeats is very different. The window-based segmentation also ensures that each window contains the same temporal and morphological structures, regardless of the underlying variations in the ECG features across the different patients. For example, a healthy patient and the patient with the PVC arrhythmias may have different heart rates, the amplitudes, and the shape of the ECG signal 134. However, by using fixed-length windows, the data of both patients may be transformed into a comparable structure, ensuring that the CESN 140 processes the data effectively without being misled by these variations.

Additionally, in the architecture 160 of the CESN 140, the reservoir 164 represents the dynamic, high-dimensional state space formed based on the ECG features. Since the input data (i.e., ECG features) is structured and consistent across the patients due to the fixed window size, the reservoir 164 may process and encode the temporal and dynamic patterns of the ECG signal 134. By using the fixed window-based segmentation method, the CESN 140 may learn and generalize better across various patients. The CESN 140 may recognize the normal heartbeats and the arrhythmic patterns such as PVCs by focusing on the structured input data, making it less sensitive to variations between the ECG features of different individuals. For example, if a new patient with the PVC arrhythmias is introduced to the system 100, the CESN 140 may already have learned the relevant features of the normal heartbeats and the abnormal heartbeats from training data. The window-based segmentation ensures that the ECG features of the new patient are analyzed in the same way as the training data, improving classification accuracy and generalization.

The training process of the CESN 140 includes data preparation, reservoir initialization, output layer training, and validation. The data preparation is a first step in the training process, where the ECG features are extracted from the raw ECG signal 134 using the modified Pan and Tompkins algorithm 138. In a preferred embodiment, the extracted ECG features may be the R-R intervals and the QRS complex. The extracted ECG features may be used as the input for the CESN 140, that provides critical temporal and morphological information about the heartbeat.

Further, the reservoir initialization is a second step in the training process where the reservoir 164 is initialized by assigning random weights to the connections between the neurons of the reservoir 164. In an embodiment, the weights may remain fixed throughout the training process to maintain the stability of the reservoir 164 and ensure efficient transformation of the input signal (i.e., ECG features) into the high-dimensional state space. The reservoir initialization ensures that the reservoir 164 captures both short-term and long-term temporal dependencies in the ECG features. As the ECG features are fed into the reservoir 164, the neurons of the reservoir 164 update their states dynamically based on the ECG features and the connections between the neurons. The reservoir 164 transforms the ECG features into the high-dimensional space, enabling the CESN 140 to encode complex temporal patterns and interdependencies in the ECG signal 134.

The output layer training is a third step in the training process where the output layer 166 is trained by using the supervised learning. The output layer 166 is only trainable layer of the CESN 140. Using the supervised learning, the weights of the output layer 166 may be optimized by using a ridge regression technique to map the states of the reservoir 164 to the desired output, such as the classification of the heartbeats. In an embodiment, the ridge regression technique is applied in the training of the CESN 140 to decrease overfitting. As used herein, “the ridge regression technique” is a regularization technique that penalizes large weights in a network, thereby controlling the complexity of the CESN 140 and enhancing the ability of the CESN 140 to generalize new data.

In an embodiment, the CESN 140 may employ sparse connectivity, which limits the number of active connections in a neural network and ensures that the CESN 140 is computationally efficient while still being able to classify the PVC heartbeats accurately.

The validation is a fourth step in the training process where the CESN 140 undergoes the tenfold cross-validation, which divides the dataset into ten subsets. The CESN 140 is trained on nine subsets and tested on the remaining one, ensuring robust performance across various datasets and reducing the risk of overfitting. The CESN 140 is trained for 10 epochs, balancing a training time with performance optimization.

The hyper-tuning is a fifth step in the training process, where hyperparameters of the CESN 140 are fine-tuned to optimize the performance. The hyperparameters may include an input dimension (i.e., number of the ECG features extracted from the ECG signal 134), a reservoir dimension (i.e., number of neurons in the reservoir 164), a readout layer dimension (i.e., number of output nodes), an input radius, an input density, a reservoir density, a reservoir radius, a feedback radius, a reservoir spectral radius (that governs the stability of the states of the reservoir 164), a leaking rate (controls how fast the reservoir 164 neurons update their states), a feedback density (determines sparsity of connections), normalization constant and bias (i.e., adjusts input scaling and bias for better generalization).

After training, the CESN 140 may be integrated into the system 100 to classify each incoming heartbeat immediately after the feature extraction. The reservoir 164 processes the ECG features using the fixed weights, ensuring low computational cost and high-speed performance. In an embodiment, the CESN 140 may be integrated into the modified Pan-Tompkins algorithm 138 to enhance the accuracy in the feature extraction. This integration ensures reliable identification of critical ECG features, such as the R-peaks and the QRS complexes, even in noisy or challenging datasets. The CESN 140 is optimized to minimize processing delays, making it suitable for real-time medical applications. This includes reducing the complexity of computations and ensuring efficient memory usage. Although the weights of the reservoir 164 remain fixed, the output layer 166 may be retrained when the new data becomes available. This adaptability ensures that the CESN 140 remains accurate and effective as new patterns or variations in the ECG signal 134 are encountered.

In an exemplary scenario, the CESN 140 processes the ECG signal 134 in real-time to detect the PVC arrhythmias. For instance, an incoming ECG signal 134 is segmented into the windows around the R-peaks using the modified Pan-Tompkins algorithm 138. The ECG features like the R-R intervals and the QRS complex are extracted and fed into the CESN 140. The reservoir 164 transforms the extracted ECG features into a high-dimensional representation, preserving the temporal patterns such as the heart rate variability. The output layer 166 classifies the heartbeat as either the normal heartbeat or the PVC heartbeat, providing immediate feedback to the healthcare team. By combining advanced feature extraction, high-dimensional encoding, and efficient real-time processing, the CESN 140 ensures the reliable detection of the PVC arrhythmias with minimal latency, even in imbalanced datasets.

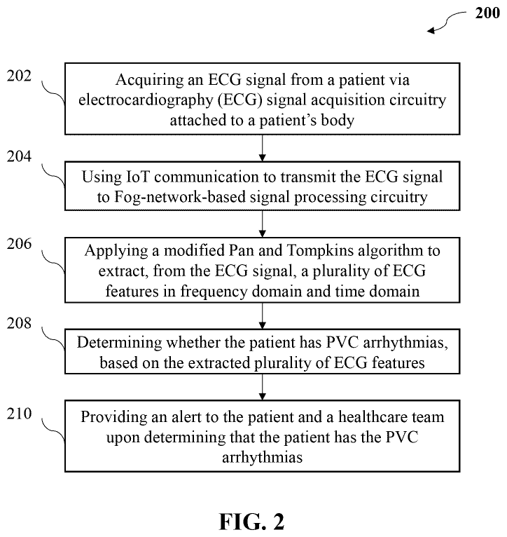

FIG. 2 illustrates a flowchart of a method 200 for performing the IoT-based detection of the PVC arrhythmias, according to certain embodiments. The method 200 includes a series of steps. These steps are only illustrative, and other alternatives may be considered where one or more steps are added, one or more steps are removed, or one or more steps are provided in a different sequence without departing from the scope of the present disclosure.

At step 202, the method 200 includes acquiring the ECG signal 134 from the patient through the ECG signal acquisition circuitry 102 attached to the patient's body 108. This step includes detecting the electrical activity of the heart by picking up the electrical impulses (i.e., the weak electrical signals) generated during depolarization and repolarization of the heart muscles using the electrodes 112. This step further includes transmitting the weak electrical signals to the AD8232-module-based sensor 110, where the weak electrical signals are processed and displayed as the ECG signal 134.

At step 204, the method 200 includes using the IoT communication, via the IoT-based signal transmission circuitry 104, to transmit the ECG signal 134 to the Fog-network-based signal processing circuitry 106. This step includes processing, by the Arduino-Nano-3-module-based controller 114, the ECG signal 134 to make the ECG signal 134 compatible with the IoT transmission. This step further includes transmitting, by the Arduino-Nano-3-module-based controller 114, the processed ECG signal 134 to the ESP8266 wireless communication component 116 using the serial communication protocol through the digital pins 2 and 3. This step further includes transmitting, by the Arduino-Nano-3-module-based controller 114, the processed ECG signal 134 wirelessly to the Fog-network-based signal processing circuitry 106 using the IoT communication protocols.

At step 206, the method 200 includes applying, via the Fog-network-based signal processing circuitry 106, the modified Pan and Tompkins algorithm 138 to extract, from the processed ECG signal 134, the ECG features in the frequency domain and time domain. This step includes using the moving integrating circuitry 152 to extract the ECG features in the frequency domain and time domain. The extracted ECG features include the QRS complex durations, the R-peaks, the R-R intervals, the average of the R-R intervals, and the average deviation of the R-R intervals.

The method 200 also includes performing the signal preprocessing on the raw ECG signal 134 before extracting the ECG features. The signal preprocessing includes the signal normalization and the signal filtering. The signal preprocessing includes rescaling the ECG signal 134 received from the IoT-based signal transmission circuitry 104 to obtain the normalized ECG signal 134, and the magnitude of the normalized ECG signal 134 falls within the predefined ECG signal magnitude range. The signal filtering includes using the median filter 144 to remove the baseline drifting from the normalized ECG signal 134. The signal filtering also includes using the FIR filter 146 to remove the power line interference from the normalized ECG signal 134.

At step 208, the method 200 includes determining, via the Fog-network-based signal processing circuitry 106, whether the patient has the PVC arrhythmias, based on the extracted ECG features. This step includes inputting the extracted ECG features into the CESN 140 trained to classify the PVC heartbeats and the normal heartbeats. In an embodiment, the CESN 140 may be trained by using the supervised learning technique. This step further includes obtaining, from the CESN 140, the output 142 representing the determination of whether the patient has the PVC arrhythmia. In an embodiment, the CESN 140 processes the ECG features and generates the binary classification output.

At step 210, the method 200 includes providing the alert to the patient and the healthcare team upon determining that the patient has the PVC arrhythmias. In an embodiment, the alert may be an audio alert, a visual alert, a digital alert, and so forth. This step includes alerting the patient and the healthcare team through the IoMT-connected devices.

The first embodiment is illustrated with respect to FIG. 1A-FIG. 1D. The first embodiment discloses the system 100 for performing Internet-of-Things (IoT)-based detection of Premature Ventricular Contractions (PVC) arrhythmias. The system 100 includes electrocardiography (ECG) signal acquisition circuitry 102. The system 100 further includes IoT-based signal transmission circuitry 104. The system 100 further includes Fog-network-based signal processing circuitry 106. The ECG signal acquisition circuitry 102 is configured to be attached to a patient's body 108 to acquire an ECG signal 134 from the patient. The IoT-based signal transmission circuitry 104 is configured to use IoT communication to transmit the ECG signal 134 to the Fog-network-based signal processing circuitry 106. The Fog-network-based signal processing circuitry 106 is configured to use a Fog network 118 to apply a modified Pan and Tompkins algorithm 138 to extract, from the ECG signal 134, a plurality of ECG features in frequency domain and time domain. The Fog-network-based signal processing circuitry 106 is further configured to use the Fog network 118 to determine whether the patient has PVC arrhythmias, based on the extracted plurality of ECG features. The Fog-network-based signal processing circuitry 106 is further configured to use the Fog network 118 to provide an alert to the patient and a healthcare team upon determining that the patient has the PVC arrhythmias. The extracted plurality of ECG features includes QRS complex durations, R-peaks, R-R intervals, an average of the R-R intervals, and an average deviation of the R-R intervals.

In an aspect, the Fog network 118 is further configured to, before extracting the plurality of ECG features, perform signal preprocessing on the ECG signal 134 to obtain a preprocessed ECG signal 134, and the signal preprocessing includes signal normalization and signal filtering.

In an aspect, the signal normalization includes rescaling the ECG signal 134 received from the IoT-based signal transmission circuitry 104 to obtain a normalized ECG signal 134, and a magnitude of the normalized ECG signal 134 falls within a predefined ECG signal magnitude range.

In an aspect, the predefined ECG signal magnitude range is [0, 1] or [−1, 1].

In an aspect, the signal filtering includes using a median filter 144 to remove baseline drifting from the normalized ECG signal 134.

In an aspect, the signal filtering includes using a finite impulse response (FIR) 146 filter to remove power line interference from the normalized ECG signal 134.

In an aspect, the Fog network 118 is further configured to: use differentiation circuitry 148 to extract, from the preprocessed ECG signal 134, gradient data used to identify borders of QRS complex areas.

In an aspect, the differentiation circuitry 148 is further configured to derive high slope values by calculating a plurality of derivative points based on a transfer function.

In an aspect, the Fog network 118 is further configured to: use squaring circuitry 150 to perform non-linear amplification on the extracted gradient data.

In an aspect, the Fog network 118 is further configured to: using moving integrating circuitry 152 to extract the plurality of ECG features in frequency domain and time domain.

In an aspect, the Fog network 118 is further configured to: input the extracted plurality of ECG features into a cyclic echo state network 140 trained for classifying PVC heartbeats and normal heartbeats. The Fog network 118 is further configured to: obtain from the cyclic echo state network 140, a determination result of whether the patient has the PVC arrhythmias.

In an aspect, ridge regression is applied in training of the cyclic echo state network 140 to decrease overfitting.

In an aspect, the ECG signal acquisition circuitry 102 includes an AD8232-module-based sensor 110 and electrodes 112 attached to limbs of the patient.

In an aspect, the IoT-based signal transmission circuitry 104 includes an Arduino-Nano-3-module-based controller 114 and an ESP8266 wireless communication component 116.

The second embodiment is illustrated with respect to FIG. 2. The second embodiment discloses the method 200 for performing Internet-of-Things (IoT)-based detection of Premature Ventricular Contractions (PVC) arrhythmias. The method 200 includes via electrocardiography (ECG) signal acquisition circuitry 102 attached to a patient's body 108, acquiring an ECG signal 134 from the patient. The method 200 further includes via IoT-based signal transmission circuitry 104, using IoT communication to transmit the ECG signal 134 to Fog-network-based signal processing circuitry 106. The method 200 further includes via the Fog-network-based signal processing circuitry 106: apply a modified Pan and Tompkins algorithm 138 to extract, from the ECG signal 134, a plurality of ECG features in frequency domain and time domain. The method 200 includes via the Fog-network-based signal processing circuitry 106: determine whether the patient has PVC arrhythmias, based on the extracted plurality of ECG features. The method 200 includes via the Fog-network-based signal processing circuitry 106: provide an alert to the patient and a healthcare team upon determining that the patient has the PVC arrhythmias. The extracted plurality of ECG features includes QRS complex durations, R-peaks, R-R intervals, an average of R-R intervals, and an average deviation of R-R intervals.

In an aspect, before extracting the plurality of ECG features, signal preprocessing is performed on the ECG signal 134, and the signal preprocessing includes signal normalization and signal filtering.

In an aspect, the signal normalization includes rescaling the ECG signal 134 received from the IoT-based signal transmission circuitry 104 to obtain a normalized ECG signal 134, and a magnitude of the normalized ECG signal 134 falls within a predefined ECG signal magnitude range.

In an aspect, the signal filtering includes using a median filter 144 to remove baseline drifting from the normalized ECG signal 134.

In an aspect, the signal filtering includes using a finite impulse response (FIR) filter 146 to remove power line interference from the normalized ECG signal 134.

In an aspect, the method 200 includes inputting the extracted plurality of ECG features into a cyclic echo state network 140 trained to classify PVC heartbeats and normal heartbeats. The method 200 includes obtaining from the cyclic echo state network 140, an output 142 representing a determination whether the patient has the PVC arrhythmia.

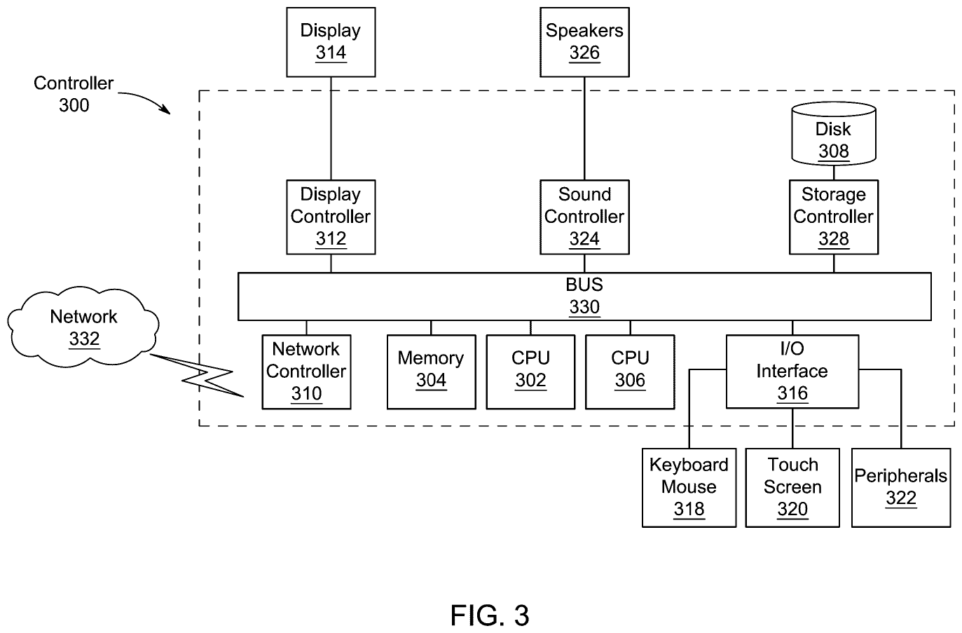

Next, further details of the hardware description of the computing environment according to exemplary embodiments are described with reference to FIG. 3. In FIG. 3, a controller 300 is described as representative of the system 100 of FIG. 1A in which the controller 300 includes a CPU 302 which performs the processes described above/below. The process data and instructions may be stored in a memory 304. These processes and instructions may also be stored on a storage medium disk 308 such as a hard drive (HDD) or a portable storage medium or may be stored remotely.

Further, claims are not limited by the form of the computer-readable media on which the instructions of the inventive process are stored. For example, the instructions may be stored on compact discs (CDs), digital versatile disc (DVDs), in FLASH memory, read access memory (RAM), read only memory (ROM), programmable read only memory (PROM), erasable programmable read only memory (EPROM), electrically erasable programmable read only memory (EEPROM), hard disk or any other information processing device with which the computing device communicates, such as a server or computer.

Further, the claims may be provided as a utility application, background daemon, or component of an operating system, or combination thereof, executing in conjunction with CPU 302, 306 and an operating system such as Microsoft Windows 7, Microsoft Windows 10, UNiplexed Information Computing System (UNIX), Solaris, Lovable Intellect Not Using XP (LINUX), Apple Macintosh (MAC)-Operating System (OS) and other systems known to those skilled in the art.

The hardware elements in order to achieve the computing device may be realized by various circuitry elements, known to those skilled in the art. For example, CPU 302 or CPU 306 may be a Xenon or Core processor from Intel of America or an Opteron processor from advanced micro devices (AMD) of America, or may be other processor types that would be recognized by one of ordinary skill in the art. Alternatively, the CPU 302, 306 may be implemented on a field programmable Gate array (FPGA), application-specific integrated circuit (ASIC), programmable logic device (PLD) or using discrete logic circuits, as one of ordinary skill in the art would recognize. Further, CPU 302, 306 may be implemented as multiple processors cooperatively working in parallel to perform the instructions of the inventive processes described above.

The computing device in the FIG. 3 also includes a network controller 300, such as an Intel Ethernet PRO network interface card from Intel Corporation of America, for interfacing with network 332. As can be appreciated, the network 332 can be a public network, such as the Internet, or a private network such as a local area network (LAN) or a wide area network (WAN) network, or any combination thereof and can also include public switched telephone network, (PSTN) or an integrated services digital network (ISDN) sub-network. The network 332 can also be wired, such as an Ethernet network, or can be wireless such as a cellular network including EDGE, 3G and 4G wireless cellular systems. The wireless network can also be Wireless Fidelity (WiFi), Bluetooth, or any other wireless form of communication that is known.

The computing device further includes a display controller 312, such as a NVIDIA GeForce GTX or Quadro graphics adaptor from NVIDIA Corporation of America for interfacing with display 314, such as a Hewlett Packard HPL2445w LCD monitor. A general purpose I/O interface 316 interfaces with a keyboard and/or mouse 318 as well as a touch screen panel 320 on or separate from display 314. General purpose I/O interface also connects to a variety of peripherals 322 including printers and scanners, such as an OfficeJet or DeskJet from Hewlett Packard.

A sound controller 324 is also provided in the computing device such as Sound Blaster X-Fi Titanium from Creative, to interface with speakers/microphone 326 thereby providing sounds and/or music.

The general-purpose storage controller 328 connects the storage medium disk 308 with communication bus 330, which may be an instruction set architecture (ISA), extended industry standard architecture (EISA), video electronics standards association (VESA), peripheral component interconnect (PCI), or similar, for interconnecting all of the components of the computing device. A description of the general features and functionality of the display 314, keyboard and/or mouse 318, as well as the display controller 312, storage controller 328, network controller 310, sound controller 324, and general purpose I/O interface 316 is omitted herein for brevity as these features are known.

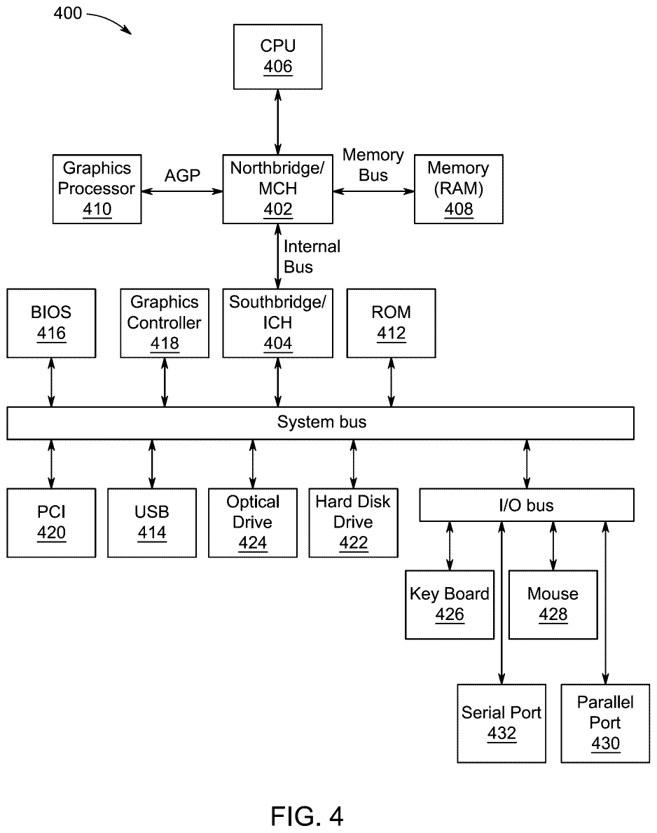

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 4.

FIG. 4 is an exemplary schematic diagram of a data processing system 400 used within the computing system, according to certain embodiments, for performing the functions of the exemplary embodiments. The data processing system 400 is an example of a computer in which code or instructions implementing the processes of the illustrative embodiments may be located.

In the FIG. 4, the data processing system 400 employs a hub architecture including a north bridge and memory controller hub (NB/MCH) 402 and a south bridge and input/output (I/O) controller hub (SB/ICH) 404. The central processing unit (CPU) 406 is connected to the NB/MCH 402. The NB/MCH 402 also connects to the memory 408 via a memory bus, and connects to the graphics processor 410 via an accelerated graphics port (AGP). The NB/MCH 402 also connects to the SB/ICH 404 via an internal bus (e.g., a unified media interface or a direct media interface). The CPU 406 may contain one or more processors and even may be implemented using one or more heterogeneous processor systems.

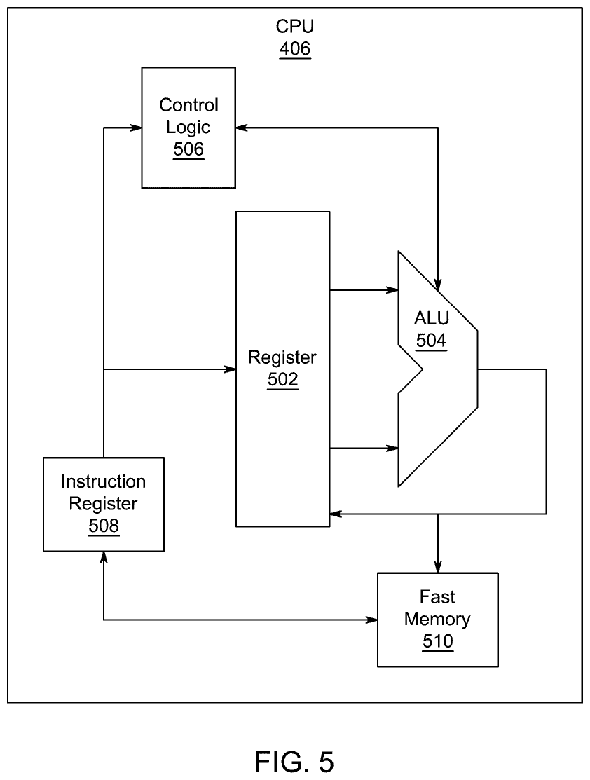

For example, FIG. 5 shows one implementation of the CPU 406. In one implementation, the instruction register 508 retrieves instructions from the fast memory 510. At least part of these instructions is fetched from the instruction register 508 by the control logic 506 and interpreted according to the instruction set architecture of the CPU 406. Part of the instructions can also be directed to the register 502. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using the arithmetic logic unit (ALU) 504 that loads values from the register 502 and performs logical and mathematical operations on the loaded values according to the instructions. The results from these operations can be feedback into the register 502 and/or stored in the fast memory 510. According to certain implementations, the instruction set architecture of the CPU 406 can use a reduced instruction set architecture, a complex instruction set architecture, a vector processor architecture, a very large instruction word architecture. Furthermore, the CPU 406 can be based on a Von Neuman model or a Harvard model. The CPU 406 can be a digital signal processor, the FPGA, the ASIC, the PLA, a PLD, or a CPLD. Further, the CPU 406 can be an x86 processor by Intel or by AMD; an ARM processor, a Power architecture processor by, e.g., IBM; a SPARC architecture processor by Sun Microsystems or by Oracle; or other known CPU architecture.

Referring again to the FIG. 4, the data processing system 400 can include that the SB/ICH 404 is coupled through a system bus to an I/O Bus, a read only memory (ROM) 412, universal serial bus (USB) port 414, a flash binary input/output system (BIOS) 416, and a graphics controller 418. PCI/PCIe devices can also be coupled to SB/ICH 404 through a PCI bus 420.

The PCI devices may include, for example, Ethernet adapters, add-in cards, and PC cards for notebook computers. The Hard disk drive 422 and CD-ROM (optical drive) 424 can use, for example, an integrated drive electronics (IDE) or serial advanced technology attachment (SATA) interface. In one implementation the I/O bus can include a super I/O (SIO) device.

Further, the hard disk drive (HDD) 422 and optical drive 424 can also be coupled to the SB/ICH 404 through a system bus. In one implementation, a keyboard 426, a mouse 428, a parallel port 430, and a serial port 432 can be connected to the system bus through the I/O bus. Other peripherals and devices that can be connected to the SB/ICH 404 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

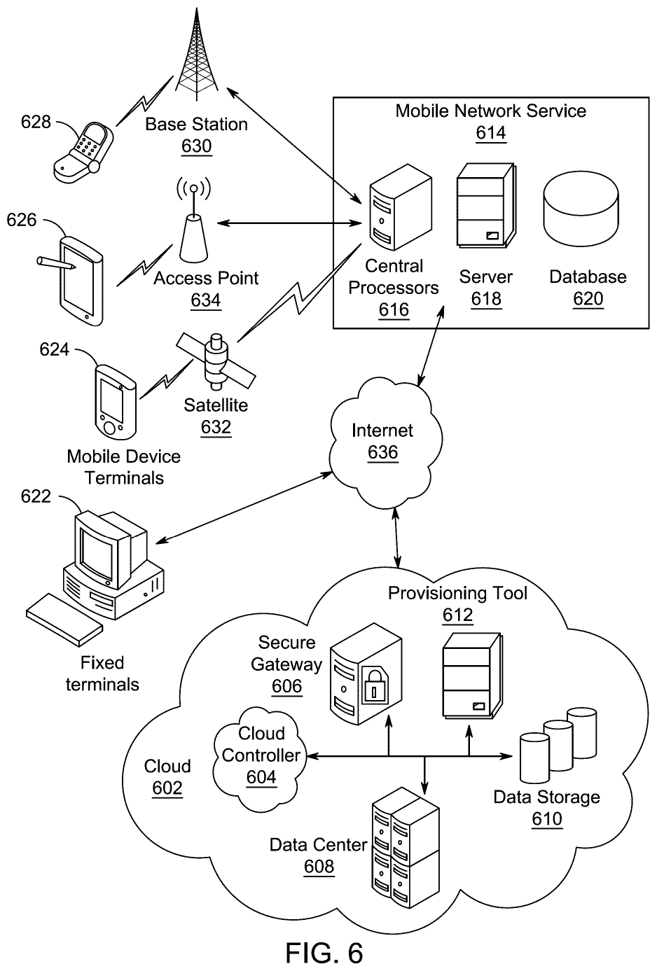

The functions and features described herein may also be executed by various distributed components of a system. For example, one or more processors may execute these system functions, wherein the processors are distributed across multiple components communicating in a network. The distributed components may include one or more client and server machines, such as cloud 602 including a cloud controller 604, a secure gateway 606, a data center 608, data storage 610 and a provisioning tool 612, and mobile network services 614 including central processors 616, a server 618 and a database 620, which may share processing, as shown by FIG. 6, in addition to various human interface and communication devices (e.g., display monitors 622, smart phones 624, tablets 626, personal digital assistants (PDAs) 628). The network may be a private network, such as a base station 630, satellite 632 or access point 634, or be a public network, may such as the Internet 636. Input to the system may be received via direct user input and received remotely either in real-time or as a batch process. Additionally, some implementations may be performed on modules or hardware that are not identical to those described. Accordingly, other implementations are within the scope that may be claimed.

The above-described hardware description is a non-limiting example of corresponding structure for performing the functionality described herein.

Numerous modifications and variations of the present disclosure are possible in light of the above teachings. It is therefore to be understood that the invention may be practiced otherwise than as specifically described herein.

1. A system for performing Internet-of-Things (IoT)-based detection of Premature Ventricular Contractions (PVC) arrhythmias, comprising:
electrocardiography (ECG) signal acquisition circuitry;
IoT-based signal transmission circuitry; and
Fog-network-based signal processing circuitry, wherein
the ECG signal acquisition circuitry is configured to be attached to a patient's body to acquire an ECG signal from the patient,
the IoT-based signal transmission circuitry is configured to use IoT communication to transmit the ECG signal to the Fog-network-based signal processing circuitry,
the Fog-network-based signal processing circuitry is configured to use a Fog network to:
apply a modified Pan and Tompkins algorithm to extract, from the ECG signal, a plurality of ECG features in frequency domain and time domain,
determine whether the patient has PVC arrhythmias, based on the extracted plurality of ECG features, and
upon determining that the patient has the PVC arrhythmias, provide an alert to the patient and a healthcare team, and
the extracted plurality of ECG features include QRS complex durations, R-peaks, R-R intervals, an average of the R-R intervals, and an average deviation of the R-R intervals.

2. The system of claim 1, wherein the Fog network is further configured to, before extracting the plurality of ECG features, perform signal preprocessing on the ECG signal to obtain a preprocessed ECG signal, and the signal preprocessing includes signal normalization and signal filtering.

3. The system of claim 2, wherein the signal normalization includes rescaling the ECG signal received from the IoT-based signal transmission circuitry to obtain a normalized ECG signal, and a magnitude of the normalized ECG signal falls within a predefined ECG signal magnitude range.

4. The system of claim 3, wherein the predefined ECG signal magnitude range is [0, 1] or [−1, 1].

5. The system of claim 3, wherein the signal filtering includes using a median filter to remove baseline drifting from the normalized ECG signal.

6. The system of claim 3, wherein the signal filtering includes using a finite impulse response (FIR) filter to remove power line interference from the normalized ECG signal.

7. The system of claim 2, wherein the Fog network is further configured to:
use differentiation circuitry to extract, from the preprocessed ECG signal, gradient data used to identify borders of QRS complex areas.

8. The system of claim 7, wherein the differentiation circuitry is further configured to derive high slope values by calculating a plurality of derivative points based on a transfer function.

9. The system of claim 7, wherein the Fog network is further configured to:
use squaring circuitry to perform non-linear amplification on the extracted gradient data.

10. The system of claim 1, wherein the Fog network is further configured to:
using moving integrating circuitry to extract the plurality of ECG features in frequency domain and time domain.

11. The system of claim 1, wherein the Fog network is further configured to:
input the extracted plurality of ECG features into a cyclic echo state network trained for classifying PVC heartbeats and normal heartbeats, and
obtain, from the cyclic echo state network, a determination result of whether the patient has the PVC arrhythmias.

12. The system of claim 11, wherein ridge regression is applied in training of the cyclic echo state network to decrease overfitting.

13. The system of claim 1, wherein the ECG signal acquisition circuitry includes an AD8232-module-based sensor and electrodes attached to limbs of the patient.

14. The system of claim 1, wherein the IoT-based signal transmission circuitry includes an Arduino-Nano-3-module-based controller and an ESP8266 wireless communication component.

15. A method for performing Internet-of-Things (IoT)-based detection of Premature Ventricular Contractions (PVC) arrhythmias, comprising:
via electrocardiography (ECG) signal acquisition circuitry attached to a patient's body, acquiring an ECG signal from the patient;
via IoT-based signal transmission circuitry, using IoT communication to transmit the ECG signal to Fog-network-based signal processing circuitry; and
via the Fog-network-based signal processing circuitry:
apply a modified Pan and Tompkins algorithm to extract, from the ECG signal, a plurality of ECG features in frequency domain and time domain,
determine whether the patient has PVC arrhythmias, based on the extracted plurality of ECG features, and
upon determining that the patient has the PVC arrhythmias, provide an alert to the patient and a healthcare team,
wherein the extracted plurality of ECG features include QRS complex durations, R-peaks, R-R intervals, an average of R-R intervals, and an average deviation of R-R intervals.

16. The method of claim 15, wherein before extracting the plurality of ECG features, signal preprocessing is performed on the ECG signal, and the signal preprocessing includes signal normalization and signal filtering.

17. The method of claim 16, wherein the signal normalization includes rescaling the ECG signal received from the IoT-based signal transmission circuitry to obtain a normalized ECG signal, and a magnitude of the normalized ECG signal falls within a predefined ECG signal magnitude range.

18. The method of claim 17, wherein the signal filtering includes using a median filter to remove baseline drifting from the normalized ECG signal.

19. The method of claim 17, wherein the signal filtering includes using a finite impulse response (FIR) filter to remove power line interference from the normalized ECG signal.

20. The method of claim 15, further comprising:
inputting the extracted plurality of ECG features into a cyclic echo state network trained to classify PVC heartbeats and normal heartbeats, and
obtaining, from the cyclic echo state network, an output representing a determination whether the patient has the PVC arrhythmia.
